(19) United States
(12) Patent Application Publication
(10) Pub. No.: US 2004/0235719 A1
(43) Pub. Date: Nov. 25, 2004

(54) Soluble protein-polymer systems for drug delivery

(76) Inventors: Roy Harris, Nottingham (GB); Paul O'Shea, Nottingham (GB)

Correspondence Address: Edwin V Merkel, Nixon Peabody, Clinton Square, P O Box 31051, Rochester, NY, 14603-1051, US

(21) Appl. No.: 10/476,784

(22) PCT Filed: May 10, 2002
(86) PCT No.: PCT/GB02/02158

(51) Int. Cl. A61K 38/38

(52) U.S. Cl. 514/12; 530/363

(57) ABSTRACT

A protein polymer is soluble in aqueous media and retains the native ligand binding properties of the protein. The polymer comprises two or more protein monomer units, such as albumin molecules, that are covalently bound through coupling agents that give rise to spacer chains of atoms between the protein molecules. The spacer chains preferably have a length of at least twenty atoms. The protein polymers are suitable for pharmaceutical drug delivery of physiologically active molecules by a variety of routes.

This invention relates to the field of drug delivery, and in particular to methods and compositions for aiding the delivery and action of physiologically active agents and in particular biologically active proteins and peptides. The invention describes the use of aqueous soluble protein carrier systems that retain their unique ligand binding properties for the attachment of biologically active therapeutic or diagnostic agents.

Drug delivery relies heavily on the carrier system for the drug providing controlled drug release at a specific time and to a targeted biological site. Many constraints are imposed on the design and use of drug delivery systems. These include the need to use materials that will receive regulatory approval as being safe and efficacious and are suitable for administration by the desired route for drug delivery (parenteral, pulmonary; nasal, oral, transdermal etc).

Many carrier systems are based on particulate polymers of the nano- or micrometer size in the form of spheres, capsules or vesicles. Many processes for producing these delivery systems create insoluble or emulsion (lipid) type carriers that alter the conformation or properties of the starting materials and often reduce their usefulness for drug interaction, targeting and release. It would be desirable to create a polymer system where the starting material is maintained in its original form, ie not excessively chemically crosslinked, heat stabilised or conformationally challenged. Also the polymer formed should present itself in the body as near to its monomeric nature as possible and not be antigenic or toxic to the patient.

The chemical crosslinking of proteins and other drugs to albumin in order to increase circulatory half-life is well documented. These methods generally involve the use of general crosslinking agents such as glutaraldehyde or ethyldimethylaminopropyl carbodiimide (EDC) relying on the side chain properties of the amino acid residues within the protein (—NH2, —COOH etc). They are restricted in terms of payload to the loading capability of monomeric protein carrier systems.

A second approach uses insoluble protein carrier systems such as albumin nano- or micro-particles or microspheres or aggregated forms of the protein. These are stabilised by the use of crosslinking agents, solvent precipitation, changes in pH or heat fixation in order to render the particles insoluble. In most cases the amino acid side chains are intact and can be used to attach bioactive compounds. However, the effect on the protein structure is varied and can lead to diverse conformational changes, degradation or unwanted polymerisation effects. These can lead to rapid removal of the particles by scavenger systems in the body or detrimental immunological effects. These effects can be seen with a wide range of proteins.

The above approaches may be harmful to labile bioactive drugs and proteins. Even when a percentage of the therapeutic agent remains active this may make the process commercially non-viable because of the initial cost of the drug in question.

Further problems with insoluble protein carrier systems are that they may not be readily degraded and in high concentration, especially for those particles or aggregates above 5-6 μm in size, could lead to potential blockage of blood capillaries, particularly those of the lung.

There have now been devised protein polymers that overcome or substantially mitigate the above-mentioned and/or disadvantages of the prior art.

According to the invention, there is provided a protein polymer that is soluble in aqueous media and retains the native ligand binding properties of the protein and is suitable for pharmaceutical drug delivery of physiologically active molecules.

By “protein polymer” is meant a molecular entity comprising two or more protein moieties that are covalently bound together. Each protein moiety thus constitutes a “monomer unit”. The number of “monomer units” that make up the “protein polymer” will generally be quite small (at least in relation to the number of monomer units making up a conventional natural or synthetic polymer), typically from 2 to 20, and the “protein polymer” may therefore be considered to be an “oligomer”. Notwithstanding this, however, because the molecular weight of each “monomer unit” may be quite substantial, the molecular weight of the “protein polymer” may be relatively large.

This invention thus relates to the formation and use of soluble protein polymers as drug delivery vehicles and means of attaching bioactive materials to the polymers by utilising specific binding properties of the protein in the polymer. The method of polymer production and drug attachment is such as to assist in maintaining the activity of the attached drug and enhance its efficacy through improved delivery. Drug delivery vehicles comprising protein polymers according to the invention may be suitable for administration by a variety of routes, eg topical or parenteral administration.

This invention relates in the first instance to the formation of soluble protein polymers. The formation of soluble polymers is achieved by controlled crosslinking between protein monomers in such a way as to maintain any required ligand-binding site intact. The definition of soluble polymer for the purposes of this invention is any non-particulate polymer system that can be prepared in aqueous solutions and that does not involve the traditional methods of particle formation (ie crystallisation and precipitation from solvents). “Soluble” in this context thus generally means soluble in water or aqueous media.

An example of a protein suitable for the formation of soluble polymer systems is albumin. Albumin has many functions, a major one being its ability to bind a number of ligands and act as a transport protein. The ligand binding properties of albumin have been studied in detail and make it an ideal candidate as a delivery system [Brown, J R and Shockley, P, (1982) Lipid-Protein Interactions, vol 1, pp25-68, ed Jost and Griffiths, Wiley; Kragh-Hansen, U (1990), Dan. Med.Bull. 37, 57-84; Peters, T, (1996), All About Albumin, pp79-132].

Clinical grade albumin as used for intravenous delivery as a blood expander (for treatment of burns patients or those suffering heavy blood loss) contains up to a permissible level of 5% polymer. A dose could be as high as 50 g which equates to an amount of polymer of up to 2.5 g. In systems according to the present invention, the level of soluble polymer to be delivered will generally fall within this range and will therefore not pose a risk to the patient.

Albumin is readily available as a fractionated product from blood plasma and is also being produced as a recombinant product. However, the invention is not restricted to albumin and other proteins could be used in a similar fashion.

Human serum albumin (HSA) is a particularly good candidate in that it contains several well characterised binding sites for a variety of molecules. The albumin molecule can be divided into three domains, each of which consists of two sub-domains. Ligands that bind to albumin can be characterised into a number of groups based on their chemical properties and generally fall into the categories of anionic or hydrophobic compounds. Recognised binding sites include long and short chain fatty acid binding regions, the binding of many drugs such as salicylate, digitoxin and warfarin at Sudlow site I (responsible for the binding of bulky heterocyclic anions with a central charge), diazepam and ibuprofen at Sudlow site II (responsible for the binding of aromatic ligands with a neutral or peripheral anionic charge), cationic drugs at residue CYS-34 and glycation of lysine residues (LYS-525, Lys-199, LYS-281, LYS-439).

If the conformational structure of the protein is maintained during the formation of the polymer then these ligand-binding sites will be available for attaching bioactive compounds. Of course the chemical side groups of the amino acid backbone will also be available for linking as in the monomeric form.

By gentle manipulation of the proteins it is possible to form “natural” linkages with an HSA polymer through well characterised binding sites.

For example, glycoproteins or drugs containing sugar residues can be attached via the glycation sites on the albumin or other proteins. Further, fatty acid residues or drug compounds or mimetics can be attached to bioactive compounds to aid the binding of these species to the fatty acid binding regions on the protein polymer. This can confer sufficiently high affinity binding to allow transport to the site of action. In the case of fatty acid binding, the non-covalent nature of the conjugation would also allow release of the drug at the site of action through, for example, controlled degradation of the polymer or transfer into cell membranes.

The maintaining of the HSA structure and function in its polymeric state also allows the carrier system to be useful in targeting albumin receptors or for the potential use in tumour targeting because of the high affinity that many tumours have for albumin.

Other proteins can also be used that contain their own binding properties (eg haemoglobin, which has porphyrin and haem binding sites and glycation sites).

In one embodiment of the current invention the polymers are formed initially by disulphide bridging between, for example, the free sulphydryl groups at CYS-34 on HSA to form dimeric species. Higher molecular weight polymers may be produced by coupling protein dimers with a bioactive compound via recognised ligand binding domains. This could take the form of a glycoprotein with several oligosaccharide side chains capable of forming bonds with adjacent dimers by glycation. Alternatively, proteins, peptides and other drugs can be labelled with multiple ligands such as salicylate or fatty acid to achieve a bridging crosslink.

In a second embodiment of this invention the protein polymers are formed by controlled coupling. An example of this is to attach sulphydryl reactive species to core protein monomers. These are then reacted either with proteins containing a single free thiol (eg CYS-34 in HSA) or to proteins containing multiple free sulphydryl groups. Inter-molecular bridging can also be enhanced by using homobifunctional coupling agent that form disulphide type bridges (eg dimaleimide reagents used in conjunction with protein thiols, heterobifunctional reagents such as iminothiolane that generate free sulphydryls on any lysine-containing protein).

This invention also relates to the formation of a polymer around a defined size lipid membrane. Lipid vesicles composed of phospholipids, modified phospholipids for attachment of proteins or a phospholipid-peptide conjugate for attachment of proteins of a defined monodispersed size, eg 100 nm, 500 nm or 1 μm, are coated with HSA or similar protein. HSA, in sufficient concentration in an aqueous buffer, will completely coat the surface of the lipid vesicle. Reactive species (—SH reagents) in the lipid membrane vesicle can be used to link HSA monomers together to form a polymeric shell of protein. Under controlled conditions the HSA protein remains in its original conformation due to limited and directed crosslinking allowing all native binding sites to be available. This allows the binding of actives to the outside of the protein shell as described below. Further actives can be encapsulated in the lipid vesicle before coating with protein, the lipid being removed by washing with an organic solvent or left in situ as part of the delivery system. In the latter case the protein would afford protection to the lipid vesicle thus overcoming many of the problems associated with liposomal drug delivery systems (eg half life time in circulation, stability).

It can be seen that this system could be modified to produce a controlled drug release system with targeting capabilities (eg wound healing where fibrinogen is used to target the carrier system for delivery of anti-scarring agent, haemostatic agents, anti-cancer agents etc). The targeting molecules (antibodies, fibrinogen) can be attached to the outside of the carrier with the drug to be delivered encapsulated on the inside. Release can be controlled by use of different levels of crosslinking of the HSA shell to allow different rates of biodegradation. Further peptide spacers can be included, to link the HSA molecules, that have labile properties and break down under certain conditions, eg pH, reduction, light, sonication etc). This can be used for example to deliver drugs to certain tumour types that are known to have cells with a reducing environment. Light energy can be used to release drugs at a specific site and is controllable from outside the patient.

Other actives that may be bound to the protein polymers of the invention include cytotoxic agents and blood clotting factors such as F-VIII and F-IX.

In another aspect, the invention relates to novel conjugation of a bioactive molecule through specific binding sites on the protein.

It is well documented that in nature the condensation of reducing sugars, such as glucose, occurs with many proteins, (Roth, M., (1983), Clin. Chem., 29, 1991). This process is referred to as glycation and can be defined as the non-enzymatic glycosylation of proteins, such as serum albumin. The principal sites of glycation are the ε-amino groups of lysine residues and the a-amino group of the protein's terminal amino acid. This reaction occurs naturally in the body under physiological conditions. Once formed, the stable ketoamine structure remains with the protein throughout its life span. Of particular relevance here is that the reaction of glucose, and other reducing sugars, with albumin involves the nucleophilic attack of the carbonyl group of the sugar at, initially, specific free amino groups on the protein (eg LYS-525 (Shaklai et al (1984) J.Biol.Chem., 259, 3812-3817)). However, in extreme conditions hyper-glycation can occur at multiple sites on the albumin molecule (Iberg and Fluckiger (1986) J. Biol. Chem., 261, 13542-13545). This principle is true of many other serum proteins.

It can be seen that a process can be defined that allows interaction between reducing sugar moieties on oligosaccharide chains of glycoproteins, or those exposed by mild enzymatic, chemical or other means, with naturally occurring linkage sites on carrier molecules through glycation. This would give an advantage over the methods that utilise general chemical crosslinking regimes in which deactivation of the active may occur or potentially toxic by-products could be produced that are difficult to remove. Also the reaction can be performed under physiological conditions that will have minimal detrimental effect on the proteins. This process can be used for conjugation of proteins to soluble or particulate protein drug delivery systems that retain their glycation binding sites.

Similarly, the attachment of, for example, salicylate (aspirin) to the bioactive compound would allow a “natural” binding of that compound to HSA polymers through recognised salicylate binding regions. The main site is the lysine residue at LYS-199 where a covalent bond would be formed. Other sites such as ARG-222 may be included.

Alternatively fatty acid residues can be conjugated to the bioactive species for attachment to the HSA polymer through fatty acid binding regions. Three main binding pockets for fatty acids are present on the HSA molecule with one or two sites being occupied at any one time. Six or more palmitate binding sites with decreasing affinities have been identified on bovine albumin and would therefore allow for multiple binding of drugs or the ability to utilise different binding regions on albumin. Typically, but not exclusively, medium to long chain fatty acids (eg oleic, palmitic, linoleic etc) or any molecule with the properties of these fatty acids could be used.

For glycoproteins these ligands could be attached through the oligosaccharide side chains using hydrazide reagents following mild periodate oxidation of the terminal sugar group. For other actives specific crosslinking regimes could be employed, depending on the bioactive being conjugated, that maintain maximal physiological activity.

The aqueous soluble protein polymer systems of the invention are suitable for parenteral and topical drug delivery and retain their ligand binding properties. The polymers may be produced as dimers or as small polymers containing between 2 and 20 protein molecules. Larger polymers can be produced by combining these smaller polymers in a controlled fashion or by utilising the combined polymers as core structure around which native protein can be attached. The core protein polymer and the coating protein need not necessarily be the same protein and combinations of different proteins could be utilised to achieve the desired drug delivery system.

The formation of polymers can be achieved in one or more steps depending on the size and use of the polymer as a drug delivery vehicle. The free sulphydryl group at CYS-34 on HSA is generally blocked and needs to be reduced before it can be utilised in polymer formation. HSA CYS-34 thiols may be reduced to free the sulphydryl groups by the addition of 10 mM dithithreitol in 20 mM phosphate buffer, pH 6, for 3-4 hours at room temperature. Low molecular weight substances may be removed by gel filtration on a Sephadex G25 column (Pharmacia PD10) in the same buffer following standard procedures or by diafiltration.

In cases where the free thiol is not required, it can be blocked to prevent unwanted crosslinking. HSA CYS-34 thiols can be blocked by the addition of 6 mole of iodoacetamide or cysteine per mole of HSA in 20 mM phosphate buffer, pH 7.5-8, for 2-5 hours at room temperature in the dark. Low molecular weight substances can be removed by gel filtration on a Sephadex G25 column (Pharmacia PD10) in the same buffer following standard procedures or by diafiltration.

The concentration of free thiols on the protein can be determined by standard procedures, eg using 10 mM dithionitrobenzoic acid (DTNB: Ellman's reagent) in 20 mM phosphate buffer, pH 8, and measuring the released TNB at 412 nm.

The formation of dimers can be achieved in various ways and can be used as building blocks for higher molecular weight polymers or as a base for bridging bioactive molecules, especially but not exclusively proteins and peptides.

One approach to forming soluble polymers involves the use of a heterobifunctional coupling agent. The coupling agent may, for instance, be reactive to thiol groups and amine groups. The coupling agent may, for example, contain maleimide groups (reactive to thiols) and succinimide ester groups (reactive to amines).

The process may be carried out in two stages, using two batches of protein. In a first stage, the coupling agent may be reacted with amine groups on the protein amino acid residue side chains. Any free thiols may first be blocked with a thiol blocking agent as described above.

In the second stage, the second batch of protein is reacted with the product of the first stage so that thiol groups in the second batch of protein react with thiol-reactive groups of the coupling agent. Blocked thiol groups may first be unblocked, eg by mild reduction as described above. Alternatively, to increase the number of thiol groups on the protein molecule a heterobifunctional reagent such as iminothiolane can be used. This reacts with amino groups on the protein amino acid residue side chains leaving exposed thiol groups on the surface of the protein. The number of thiol groups can be controlled by the relative proportion of reagent to protein used in the reaction.

Another approach to the formation of protein dimers involves the use of a homobifunctional coupling agent. Examples of suitable such agents are reagents comprising two thiol-reactive maleimide groups.

In either the first or the second approach, the reactive groups of the coupling agent (whether the same, as in a homobifunctional coupling agent, or different, as in a heterobifunctional coupling agent) will be separated by a chain of atoms that constitutes a “spacer”. The spacer may comprise a simple chain of carbon atoms (ie an alkylene chain), which may be substituted and/or interrupted by heteroatoms. Alternatively, the spacer may include one or more cyclic groups.

In both the first approach and the second approach, it is preferred that the spacer is relatively lengthy. In particular, it is preferred that the spacer constitute a chain of 20 atoms or more (some of which atoms may form part of one or more cyclic groups), more preferably more than 30 atoms, or more than 40 atoms. The length of the spacer chain may be as much as 60 atoms, or 80 atoms, or 100 atoms, or more. In terms of the physical length of the spacer chain, it is preferably greater than 25 Angstroms in length, more preferably greater than 30 Angstroms or 40 Angstroms, or longer.

Coupling agents giving rise to suitably lengthy spacer chains may be assembled by reaction of suitable intermediate compounds. For example, dithiol compounds may be used to couple together compounds containing maleimido groups.

It will be appreciated that-protein polymers comprising more than two monomer units may be formed by methods analogous to those described above.

The invention is illustrated by the following non-limiting Examples:

Addition of Iminothiolane

Thiol groups can be added to the surface of a protein by reacting lysine side chain amino groups with iminothiolane or a similar thiolating reagent. The number of thiol groups added is related to the number of available amine groups and the relative concentration of the iminothiolane to protein in the reaction mixture. All reactions were performed in 74 mM phosphate buffer pH=7.5 at room temperature in the dark for up to 1 hour. Excess reagents were removed by gel filtration (eg on Pharmacia PD10 columns) in the same buffer using standard techniques.

Reduction of HSA CYS-34

The —SH group on CYS-34 of HSA was made available by mild reduction in the presence of 10 mM dithiothreitol or a similar reducing agent followed by gel filtration to remove reagents and unbound material.

Visualisation of Polymer Formation

SDS non-reducing polyacrylamide gel electrophoresis using 5-10% acrylamide slab gels was used in a BioRad mini PROTEAN electrophoresis module following the manufacturer's instructions. Protein bands were visualised using standard Coomasie blue staining techniques.

Coupling of HSA Molecules to form Soluble Polymers

a) Reaction of Iminothiolane-Treated HSA with N-(maleimidocaproyloxy)succinimide ester (EMCS)

EMCS (0.4 ml of 3.2 mmol dissolved in DMSO) and added to 10 mg/ml non-reduced HSA in 74 mM phosphate buffer pH=7.5 in the dark at room temperature. The sample was desalted prior to addition to an equal amount of iminothiolane-treated HSA as above and incubation in the dark at room temperature overnight.

The EMCS-HSA was also reacted with different concentrations of reduced HSA under the same conditions.

Up to 40% polymer formation was observed with the addition of reduced HSA (8-10 molar excess of coupling agent to protein). This was improved upon by using iminothiolane-treated HSA (6.5 mg iminothiolane added to 50 mg protein).

Approximately 50% polymer was formed using to a 1:1 molar ratio of coupling agent to protein.

b) Long Spacer Arm M-NHS (1) (M-(A)-NHS)

M-(A)-NHS was prepared by adding equal molar ratios of EMCS in DMSO and 2-mercaptoethylether in DMSO at room temperature for 10 minutes with stirring followed by dropwise addition of an equimolar amount of bisphenylenedimaleimidomercaptoethylether (BPDME) in DMSO with stirring for 10 minutes.

The M-(A)-NHS coupling agent was added to non-reduced HSA in 74 mM phosphate buffer pH=7.5 followed by incubation for 1-2 hours at room temperature in the dark. The samples were then desalted by gel filtration as above.

Different molar ratios of coupling agent to protein (10:1 to 100:1) were used.

The M-(A)-NHS-HSA was added to different molar ratios of either iminothiolane-treated HSA (6.5 mg/50 mg protein) or reduced HSA with incubation at room temperature overnight in the dark.

The samples were electrophoresed as described above.

In both cases high yields of polymer were obtained and the percentage increased with the increase of the coupling agent concentration. The reaction with iminothiolane-treated HSA gave better yields of up to 75% polymer formation.

c) Long Spacer Arm M-NHS (2) (M-(B)-NHS)

The same experiment was performed using 1,11-bismaleimidotetraethyleneglycol (BM[PEO]₄) instead of BPDME. Similar results were obtained to those above for the M-(B)-NHS coupling agent.

d) Long Spacer Arm M-(C)-M

A long spacer arm bismaleimide coupling agent was prepared by adding equimolar amounts of BPDME and BM[PEO]₄ together in DMSO for 5 minutes. Mercaptoethylether was added to the mixture with additional stirring for 15 minutes.

Iminothiolane (6.5 mg) was added to 50 mg HSA in 1 ml 74 mM phosphate buffer pH=7.5 followed by incubation at room temperature in the dark for 1 hour. Excess reagents were removed by desalting as above. The resulting product (M-(C)-M) was added to the desalted protein in a molar ratio of 40:1-1400:1 coupling agent/protein followed by incubation for different time periods (1-8 days).

The samples were electrophoresed as described above.

Between 70% and 100% polymer could be formed by adjusting the amount of coupling agent and time period of incubation. Excessive coupling agent and/or time lead to the formation of insoluble gel like polymers that did not enter into the electrophoresis gels and could be centrifuged down to form a pellet.

1. A process for coupling together molecules of a protein to form a protein polymer that is soluble in aqueous media, which process comprises the steps of
a) reacting a first aliquot of the protein with a heterobifunctional coupling agent; and
b) reacting the product of steps a) with a second aliquot of the protein.

2. A process as claimed in claim 1, wherein the coupling reagent contains groups reactive to thiol groups and groups reactive to amine groups.

3. A process as claimed in claim 1, wherein in step a) the coupling agent reacts with amine groups on the surface of the protein and step b) involves reaction of the coupling agent with thiol groups on the surface of the protein in the second aliquot.

4. A process as claimed in claim 3, wherein the thiol groups on the surface of the first aliquot of protein are blocked with a thiol blocking agent prior to step a).

5. A process as claimed in claim 4, wherein the thiol blocking agent is selected from iodoacetamide and cysteine.

6. A process as claimed in claim 1, wherein blocked thiol groups on the surface of the second aliquot of protein are unblocked by a thiol unblocking agent prior to step b).

7. A process as claimed in claim 6, wherein the thiol unblocking agent is dithithreitol.

8. A process as claimed in claim 1, wherein the second aliquot of protein is thiolated by reaction with a thiolating reagent prior to step b).

9. A process as claimed in claim 8, wherein the thiolating reagent is iminothiolane.

10. A process as claimed in claim 1, wherein the protein is albumin.

11. A process as claimed in claim 10, wherein the protein is human serum albumin.

12. A process as claimed in claim 1, wherein the protein is recombinantly derived.

13. A process as claimed in claim 12, wherein blocked sulphydryl groups on the protein are unblocked by the addition of dithithreitol prior to step b).

14. A process as claimed in claim 1, with further comprises the step of reacting the protein polymer with a physiologically active therapeutic or diagnostic agent.

15. A process as claimed in claim 14, wherein the said agent is fibrinogen.

16. A process as claimed in claim 14, wherein the said agent is F-VIII or F-IX.

17. A process as claimed in claim 14, wherein the said agent is a cytotoxic agent.

18. A process as claimed in claim 14, wherein the said agent acts as a targeting agent.

19. A process as claimed in claim 1, wherein to protein polymer is between 1 nm and 1000 nm in diameter.

20. A process as claimed in claim 1, wherein the protein polymer comprises individual protein molecules coupled together by spacer chains having a length of at least 20 atoms.
